(19) United States
(12) Patent Application Publication
(10) Pub. No.: US 2026/0026873 A1
(43) Pub. Date: Jan. 29, 2026

(54) IMPEDANCE STOPPAGE MITIGATION DURING RADIOFREQUENCY TISSUE ABLATION PROCEDURES

(71) Applicant: Relievant Medsystems, Inc., Redwood City, CA (US)

(72) Inventors: Brian W. Donovan, San Jose, CA (US); Alexander Pruitt, San Jose, CA (US)

(21) Appl. No.: 19/349,677

(22) Filed: Oct. 3, 2025

Related U.S. Application Data
(62) Division of application No. 18/053,284, filed on Nov. 7, 2022, now Pat. No. 12,433,668.
(60) Provisional application No. 63/277,100, filed on Nov. 8, 2021.

(51) Int. Cl. A61B 18/14; A61B 18/00; A61B 18/12

(52) U.S. Cl. CPC A61B 18/148; A61B 2018/00434; A61B 2018/00577; A61B 2018/00702; A61B 2018/00714; A61B 2018/00732; A61B 2018/00755; A61B 2018/00761; A61B 2018/00791; A61B 2018/126

(57) ABSTRACT

A radiofrequency tissue ablation system is configured to reduce occurrences of impedance-related stoppages of radiofrequency energy delivery during a radiofrequency tissue ablation procedure. The system includes a radiofrequency generator and one or more processors operatively coupled to the power source and to the one or more temperature sensors. The processor is configured to cause the power source to deliver radiofrequency energy to the electrodes to provide controlled heating of tissue and receive a plurality of temperature measurements from the temperature sensors, calculate a magnitude and a rate of change of tissue impedance based on signals received from the one or more electrodes and determine whether one or more trigger criteria are met. If the one or more trigger criteria are met, the processor causes the power source to temporarily reduce power output to reduce temperature to a lower threshold temperature and to ramp power output.

### CROSS-REFERENCE TO RELATED APPLICATIONS

The present application is a divisional of U.S. patent application Ser. No. 18/053,284, filed Nov. 7, 2022, which claims priority to U.S. Provisional Application No. 63/277,100 filed Nov. 8, 2021, the entire content of which are hereby incorporated by reference.

### FIELD

Described herein are various embodiments of systems and methods for preventing, mitigating, or reducing the occurrence of impedance-related stoppages during radiofrequency procedures (e.g., radiofrequency ablation treatment procedures), such as intraosseous nerve or basivertebral nerve ablation procedures for treatment or prevention of back pain or other tissue ablation, stimulation, or other modulation or modification procedures.

### BACKGROUND

Radiofrequency energy has been used to ablate tissue at various locations within the body. For example, radiofrequency probes have been used to ablate tissue within the heart to treat atrial fibrillation. Radiofrequency probes have also been used to ablate nerves to facilitate treatment of facet joint pain or joint pain within the knees, shoulders, elbows, hips, or ankles.

### SUMMARY

One of the characteristics of radiofrequency ablation procedures is that energy transfer between the electrode(s) of a radiofrequency energy delivery device (e.g., radiofrequency probe) and tissue is dependent on sufficient electrical and thermal tissue conductivity. If power output rises quickly or power output is affected due to anatomical differences or physiological conditions such as blood flow direction, blood flow rate and vascularity at an ablation location (e.g., within a vertebral body), the tissue around the electrode can become desiccated (e.g., charred). Desiccated tissue acts as an insulating “sleeve” around the electrode(s), which limits the transmission of further electrical or thermal energy and can limit any further extension of desired tissue destruction or the ablation zone. Desiccated tissue impedance is typically high, which can lead to an automated treatment stoppage if the tissue impedance triggers pre-set threshold impedance levels, thereby causing the radiofrequency generator to automatically stop the radiofrequency ablation procedure.

Radiofrequency ablation procedures may be performed to ablate different types of tissue (including nerve tissue, adipose tissue, muscle tissue). The procedures may be performed at various different locations on an external surface of a subject or within a subject. Procedures performed within a subject may be performed in a minimally invasive manner (e.g., via a percutaneous, laparoscopic, endoscopic, or intravascular approach) or in a more invasive manner (e.g., via an open surgical procedure). For example, Applicant's existing technology (the Intracept® procedure by Relievant® Medsystems, Inc.) offers a safe and effective minimally invasive procedure that targets the basivertebral nerve for the relief of chronic low back pain that originates at least partly from one or more endplates of one or more vertebral bodies. The Intracepto procedure involves application of radiofrequency energy from a radiofrequency generator using a bipolar radiofrequency energy delivery device (e.g., probe), wherein the applied radiofrequency energy is sufficient to ablate the basivertebral nerve within a vertebral body. The procedure may be performed in multiple different vertebral bodies sequentially or simultaneously (using a single radiofrequency energy delivery device or multiple radiofrequency energy delivery devices coupled to a single generator). The basivertebral nerve trunk may be ablated. Other intraosseous nerves within the vertebral body that innervate the endplates and/or intervertebral disc may also be targeted and ablated.

During the Intracepta procedure, as tissue desiccates, the radiofrequency generator (“RFG”) monitors the rising tissue impedance during the treatment. If tissue impedance rises above the RFG's pre-set threshold impedance level (e.g., maximum tolerance level), the RFG may automatically stop radiofrequency energy delivery and halt the treatment. To continue the treatment, the physician or other clinical professional removes the radiofrequency probe from the patient, cleans the electrodes by wiping away any coagulum or char that might have formed. The physician or other clinical professional then reinserts and repositions the radiofrequency probe in the vertebral body at a target region (e.g., posterior 50% of the vertebral body). When the RFG obtains an impedance value within an acceptable range (e.g., below the pre-set threshold impedance level by a certain percentage or by a certain value), the RFG notifies the physician or other clinical professional that the system is ready to re-start radiofrequency energy delivery. Although impedance-based stoppages may be common and expected during an RF ablation, they can delay the overall duration of the procedure and frustrate the physician and/or other clinical professionals (especially if there are multiple stoppage occurrences during the same treatment procedure), further extending the time to complete the procedure.

Radiofrequency procedures may result in impedance-related stoppages based on tissue desiccation, or tissue charring. Embodiments of monitoring algorithms described herein may advantageously result in reducing the number of impedance-related stoppages (e.g., by reducing power based on monitored impedance and allowing the tissue to revascularize and/or rehydrate such that the tissue does not get to the point of charring or desiccation that results in impedance-related stoppages). In accordance with several embodiments, the systems and methods described herein advantageously prevent, mitigate, or reduce the likelihood of desiccation or charring, thereby preventing, mitigating, or reducing the likelihood of impedance-related stoppage during radiofrequency ablation procedures, which can result in shorter procedure times and reduced clinician intervention.

In accordance with several embodiments, a radiofrequency tissue ablation system configured to reduce occurrences of impedance-related stoppages of radiofrequency energy delivery during a radiofrequency tissue ablation procedure includes a radiofrequency probe comprising one or more electrodes and one or more temperature sensors. The system also includes a radiofrequency generator comprising a power source electrically coupled to the one or more electrodes and one or more processors operatively coupled to the power source and to the one or more temperature sensors. The one or more processors are configured to execute program instructions stored in memory of the radiofrequency generator that, when executed, cause the power source to deliver radiofrequency energy to the one or more electrodes, the radiofrequency energy being sufficient to provide controlled heating of tissue surrounding the one or more electrodes; and cause the radiofrequency generator to transition between a steady-state phase of operation and a triggered impedance-limiting phase of operation so as to reduce temperature and tissue impedance prior to initiation of an automated impedance-related stoppage of radiofrequency energy delivery during a radiofrequency tissue ablation procedure. In some embodiments, the radiofrequency energy generator is also configured to operate in an initial ramp phase prior to the steady state phase.

When operating in the steady-state phase, the one or more processors are configured to receive temperature measurements from the one or more temperature sensors, calculate a magnitude and a rate of change of tissue impedance based on signals received from the one or more electrodes, and automatically transition to the impedance-limiting phase of operation when one or more trigger criteria are met. When operating in the impedance-limiting phase, the one or more processors are configured to cause the power source to temporarily reduce power output to reduce temperature (e.g., to a lower threshold temperature lower than a set target temperature) and to ramp power output back up after the tissue has at least partially rehydrated for a certain duration of time (e.g., after temperature maintenance at the lower threshold temperature for a certain period of time). In some embodiments, the set target temperature is between 43 degrees Celsius and 100 degrees Celsius, between 43 degrees and 70 degrees Celsius, between 43 degrees Celsius and 67 degrees Celsius, between 50 degrees Celsius and 60 degrees Celsius, between 60 degrees Celsius and 65 degrees Celsius, between 60 degrees Celsius and 90 degrees Celsius, between 70 degrees Celsius and 100 degrees Celsius, overlapping ranges thereof, or any value within the recited ranges. The lower threshold temperature may be a value less than the set target temperature and within the same ranges of the set target temperature previously listed. The ramp rate may be between 0.01 degrees Celsius per second and 5 degrees Celsius per second (e.g., between 0.01 and 1, between 0.5 and 1.5, between 1 and 3, between 2.5 and 5, between 0.1 and 0.5, between 0.1 and 1.5, between 0.5 and 2, between 1 and 2.5, overlapping ranges thereof, or any value within the recited ranges).

In some instances, the radiofrequency probe includes two electrodes positioned along a distal end portion of the radiofrequency probe configured to operate as a bipolar pair of electrodes. The radiofrequency probe may include one or more electrodes configured to operate as a monopolar electrode. When in the steady-state phase of operation, the radiofrequency energy may have a frequency between 300 kHz and 800 kHz (e.g., between 350 kHz and 650 kHz, between 400 kHz and 600 kHz, between 500 kHz and 700 kHz, overlapping ranges thereof, or any value within the recited ranges) and may be applied for a duration sufficient to generate a controlled ablation zone to ablate tissue within the controlled ablation zone. In some embodiments, the duration is between 1 minute and 20 minutes (e.g., between 5 minutes and 15 minutes, between 5 minutes and 10 minutes, between 6 minutes and 8 minutes, between 10 minutes and 15 minutes, overlapping ranges thereof, or any value within the recited ranges).

In some embodiments, the controlled ablation zone has a maximum cross-sectional dimension of 35 mm.

In some embodiments, the controlled ablation zone has a maximum cross-sectional dimension of 30 mm.

In some embodiments, the controlled ablation zone has a maximum cross-sectional dimension of 25 mm.

In some embodiments, the controlled ablation zone has a maximum cross-sectional dimension of 20 mm, 15 mm, or 10 mm.

When in the steady-state phase of operation, a power level may be a power level sufficient to ablate tissue (e.g., between 0 and 20 Watts, greater than 0 Watts to 10 Watts, between 1 Watt and 8 Watts, between 2 Watts and 10 Watts, between 5 Watts and 15 Watts, between 8 Watts and 20 Watts, overlapping ranges thereof, or any value within the recited ranges). In the impedance-limiting phase, the power output may be reduced until a lower threshold temperature is reached. When in the impedance-limiting phase of operation, a power level may be reduced to a lower level at which impedance can be monitored but at which no significant radiofrequency energy is being delivered. The ramp up in power in the impedance-limiting phase of operation may not occur until after a dwell period once the lower threshold temperature has been reached.

In some embodiments, the signals received from the one or more electrodes are received at a certain interval (e.g., between 5 ms and 100 ms, between 5 ms and 20 ms, between 10 ms and 30 ms, between 20 ms and 40 ms, between 30 ms and 50 ms, between 40 ms and 60 ms, between 50 ms and 70 ms, between 60 ms and 80 ms, between 50 ms and 100 ms, overlapping ranges thereof, or any value within the recited ranges).

The system may include one or more access instruments configured to facilitate access to a target treatment location within a bone (e.g., a vertebral body), organ, or other external or internal target treatment location. The tissue to be ablated may be a basivertebral nerve or other intraosseous nerve within a vertebral body. The nerves may be nerves that innervate one or more endplates and/or intravertebral discs of the spine at one or more levels of the spine (e.g., cervical, thoracic, lumbar, sacral, or lumbosacral). Of course, the tissue may be another nerve or body tissue at other locations within other bones or outside of bones.

Calculation of the rate of change of tissue impedance may involve calculation of a rolling average of tissue impedance measurements over a particular time duration. The particular time duration may be between 25 ms and 20,000 ms (e.g., between 25 ms and 500 ms, between 50 ms and 1000 ms, between 500 ms and 10,000 ms, between 1000 ms and 20,000 ms, overlapping ranges thereof, or any value within the recited ranges). The particular time duration may alternatively be based on a number of impedance measurement datapoints instead of directly being based on a time duration (e.g., 5 to 200 datapoints, 5 to 50 datapoints, 20 to 100 datapoints, 50 to 150 datapoints, 100 to 200 datapoints, overlapping ranges thereof, or any value within the recited ranges). The one or more trigger criteria can include at least one criterion based on rate of change of tissue impedance and/or at least one criterion based on the temperature.

In some embodiments, the one or more trigger criteria include a predetermined upper threshold rate of change of tissue impedance.

In some embodiments, the one or more trigger criteria have to be met for a certain number of data points or for a certain duration of time before the transition to the impedance-limiting phase of operation.

The certain number of data points may be between 1 and 50 (e.g., between 1 and 15, between 5 and 15, between 10 and 20, between 10 and 25, between 15 and 30, between 15 and 45, between 20 and 50, between 25 and 50, between 30 and 50, overlapping ranges thereof, or any value within the recited ranges). The certain duration of time may be between 5 ms and 5000 ms (e.g., between 5 ms and 100 ms, between 100 ms and 500 ms, between 250 ms and 750 ms, between 500 ms and 1000 ms, between 1000 ms and 2500 ms, between 1500 ms and 3000 ms, between 2000 ms and 4000 ms, between 2500 ms and 5000 ms, between 3000 ms and 5000 ms, between 4000 ms and 5000 ms, overlapping ranges thereof, or any value within the recited ranges).

When in the impedance-limiting phase of operation, the one or more processors may also be configured to continue to receive temperature measurements from the one or more temperature sensors and signals from the one or more electrodes to be used to calculate and monitor impedance.

The one or more processors may be configured to transition the radiofrequency generator back to the steady-state phase of operation after a pre-set target temperature has been reached following the ramp up in power in the impedance-limiting phase.

The system may include multiple radiofrequency probes operatively coupled to the radiofrequency generator that are configured to operate in the steady-state phase and the impedance-limiting phase and are controlled and monitored independently by the radiofrequency generator.

In accordance with several embodiments, a radiofrequency tissue ablation system configured to reduce occurrences of impedance-related stoppages of radiofrequency energy delivery during a radiofrequency tissue ablation procedure includes a radiofrequency probe comprising a plurality of electrodes and at least one temperature sensor. The system also includes a radiofrequency generator including a power source electrically coupled to the plurality of electrodes and one or more processors operatively coupled to the power source and to the at least one temperature sensor. The one or more processors are configured to execute program instructions that, when executed: cause the power source to deliver radiofrequency energy to the plurality of electrodes, the radiofrequency energy being sufficient to provide controlled heating of tissue surrounding the plurality of electrodes; and cause the radiofrequency generator to transition between a steady-state phase of operation and an impedance-limiting phase of operation so as to reduce temperature and tissue impedance prior to initiation of an automated impedance-related stoppage of radiofrequency energy delivery during a radiofrequency tissue ablation procedure.

When operating in the steady-state phase, the one or more processors are configured to: receive temperature measurements from the at least one temperature sensor; calculate a magnitude and a rate of change of tissue impedance based on signals received from the plurality of electrodes; and automatically transition to the impedance-limiting phase of operation when one or more trigger criteria are met. The one or more trigger criteria include at least one criterion based on the rate of change of tissue impedance.

When in the impedance-limiting phase of operation, the one or more processors are configured to cause the power source to temporarily reduce power output to reduce temperature to a lower threshold temperature and to ramp power output back up after achieving the lower threshold temperature.

In some embodiments, the radiofrequency generator is configured to provide radiofrequency energy having a frequency between 400 kHz and 600 kHz and to apply the radiofrequency energy for a duration sufficient to generate a controlled ablation zone to ablate tissue within the controlled ablation zone.

In some embodiments, the calculation of the rate of change of tissue impedance involves calculation of a rolling average of tissue impedance measurements over a particular time duration.

In some embodiments, the one or more trigger criteria include at least one criterion based on temperature and/or at least one criterion based on the rate of change of tissue impedance.

In accordance with several embodiments, a computer-implemented method to reduce occurrences of impedance-related stoppages of radiofrequency energy delivery by a radiofrequency generator during a radiofrequency tissue ablation procedure includes executing stored program instructions via one or more hardware processors. The stored program instructions may be configured to cause the generator to operate in a steady-state phase of operation after an initial ramp up of power output. When operating in the steady-state phase, the one or more hardware processors are configured to, upon execution of the program instructions, cause the radiofrequency generator to deliver radiofrequency energy to one or more electrodes of a radiofrequency probe sufficient to generate a controlled heating zone sufficient to ablate tissue surrounding the one or more electrodes. The one or more processors are configured to continuously receive temperature measurements from one or more temperature sensors positioned along the radiofrequency probe and to calculate a magnitude and a rate of change of tissue impedance based on signals received from the one or more electrodes. The one or more processors are further configured to, upon execution of the stored program instructions, cause the generator to automatically transition to an impedance-limiting phase of operation when one or more trigger criteria are met based, at least in part, on the temperature measurements and/or the calculated rate of change of tissue impedance.

When operating in the impedance-limiting phase, the one or more hardware processors are configured to, upon execution of stored program instructions, cause the generator to temporarily reduce power output (e.g., to a reduced power level) to maintain temperature at a lower threshold temperature than the set target temperature and then to ramp power output back up (e.g., to increase temperature to the set target temperature) after temperature reduction at the lower threshold temperature has been maintained (e.g., after at least partially rehydrating the tissue) for a certain time duration.

The signals received from the one or more electrodes may be signals indicative of current levels. The magnitude and the rate of change of tissue impedance may be calculated based on known voltage levels and on the received current signals (e.g., by application of Ohm's law). The calculation of the rate of change of tissue impedance may involve calculation of a rolling average of tissue impedance measurements over a particular time duration. The one or more trigger criteria may include a predetermined upper threshold value for temperature and/or a predetermined upper threshold value for rate of change of tissue impedance.

The method may also include executing stored program instructions via one or more hardware processors to cause the generator to transition back to the steady-state phase of operation after a set target temperature has been reached (e.g., as determined from temperature measurements received from the one or more temperature sensors) following ramping of the power output back up after temperature reduction at the lower threshold temperature level has been maintained for a certain time duration (e.g., after temperature reduction has achieved at least partial revascularization and/or rehydration of tissue for a certain time duration). In some embodiments, the generator is prevented from re-entering the impedance limiting phase for a period of time after transitioning back to the steady-state phase.

The power output reduction upon entry of the impedance-limiting phase may comprise a reduction of power to a power level that still allows calculation of the magnitude and the rate of change of tissue impedance. When in the impedance-limiting phase of operation, the one or more processors may be configured to continue to receive temperature measurements from the one or more temperature sensors and signals from the one or more electrodes to facilitate impedance calculations.

In accordance with several embodiments, a computer-implemented method to reduce occurrences of impedance-related stoppages of radiofrequency energy delivery by a radiofrequency generator during a radiofrequency tissue ablation procedure is provided. The method includes detecting trigger criteria indicative of a likely impedance-based stoppage. The trigger criteria include a predetermined upper threshold value for temperature and a predetermined upper threshold value for rate of change of tissue impedance. The method also includes causing the radiofrequency generator to automatically transition to an impedance-limiting phase of operation when the trigger criteria are detected.

The method may be performed by executing stored program instructions via one or more hardware processors. When operating in the impedance-limiting phase of operation, the one or more hardware processors may be configured to cause the radiofrequency generator to temporarily reduce power output to reduce temperature to a lower threshold temperature and then to ramp power output back up after the lower threshold temperature has been maintained for a certain dwell time duration.

In some embodiments, the trigger criteria are detected based on signals received from one or more electrodes of a radiofrequency probe delivering the radiofrequency energy from the radiofrequency generator and from signals received from one or more temperature sensors of the radiofrequency probe.

In some embodiments, the signals are indicative of current levels and the predetermined upper threshold value for rate of change of tissue impedance is calculated based on known voltage and the received signals indicative of current levels.

In some embodiments, the predetermined upper threshold value for rate of change of tissue impedance is between 2 and 100 ohms per second squared (e.g., between 2 and 50, between 5 and 15, between 10 and 40, between 15 and 45, between 5 and 25, between 10 and 30 between 40 and 60 between 50 and 75, between 60 and 100, between 75 and 100, overlapping ranges thereof, or any value within the recited ranges).

In some embodiments, the one or more trigger criteria have to be met for a certain number of data points or for a certain duration of time before the transition to the impedance-limiting phase of operation.

The certain number of data points may be between 1 and 50 (e.g., between 1 and 15, between 5 and 15, between 10 and 20, between 10 and 25, between 15 and 30, between 15 and 45, between 20 and 50, between 25 and 50, between 30 and 50, overlapping ranges thereof, or any value within the recited ranges). The certain duration of time may be between 5 ms and 5000 ms (e.g., between 5 ms and 100 ms, between 100 ms and 500 ms, between 250 ms and 750 ms, between 500 ms and 1000 ms, between 1000 ms and 2500 ms, between 1500 ms and 3000 ms, between 2000 ms and 4000 ms, between 2500 ms and 5000 ms, between 3000 ms and 5000 ms, between 4000 ms and 5000 ms, overlapping ranges thereof, or any value within the recited ranges).

In some embodiments, the calculation of the rate of change of tissue impedance involves calculation of a rolling average of tissue impedance measurements over a particular time duration.

The particular time duration may be between 25 ms and 20,000 ms (e.g., between 25 ms and 500 ms, between 50 ms and 1000 ms, between 500 ms and 10,000 ms, between 1000 ms and 20,000 ms, overlapping ranges thereof, or any value within the recited ranges). The particular time duration may alternatively be based on a number of impedance measurement datapoints instead of directly being based on a time duration (e.g., 5 to 200 datapoints, 5 to 50 datapoints, 20 to 100 datapoints, 50 to 150 datapoints, 100 to 200 datapoints, overlapping ranges thereof, or any value within the recited ranges). The one or more trigger criteria can include at least one criterion based on rate of change of tissue impedance and/or at least one criterion based on the temperature.

In some embodiments, the method further includes causing the generator to generate an output for display indicative that the impedance-limiting phase of operation has been initiated.

In some embodiments, the methods described herein do not involve calculation of the magnitude of the tissue impedance.

In some embodiments, the radiofrequency energy delivered is not sufficient to ablate but otherwise modulates (e.g., stimulates, denervates) the tissue.

In some embodiments, the radiofrequency energy delivered does not thermally heat the tissue but effects pulsed field ablation or electroporation.

In accordance with several embodiments, a method of treating back pain (e.g., chronic low back pain) by ablating a basivertebral nerve and/or other intraosseous nerves within one or more vertebral bodies includes inserting at least a distal end portion of a radiofrequency probe within a vertebral body of a subject. The distal end of the radiofrequency probe includes one or more electrodes (e.g., one, two, three, or more than three electrodes) and one or more temperature sensors (e.g., one, two, three, or more than three temperature sensors) electrically coupled to a radiofrequency generator. The method also includes applying power from the radiofrequency generator to the one or more electrodes to deliver radiofrequency energy sufficient to ablate a basivertebral nerve within the vertebral body. The radiofrequency generator is configured to operate in a steady-state phase of operation and a triggered impedance-limiting phase of operation so as to reduce occurrences of automatic impedance-related stoppages during delivery of the radiofrequency energy delivery. The radiofrequency generator may also be configured to operate in an initial ramp phase prior to operation in the steady state phase.

The radiofrequency generator is configured to transition to the impedance-limiting phase of operation when one or more trigger criteria are met based, at least in part, on monitoring of temperature measurements received from the one or more temperature sensors and/or on monitoring of a rate of change of tissue impedance calculated based on signals received from the one or more electrodes. When in the impedance-limiting phase of operation, the radiofrequency generator temporarily reduces power output to reduce temperature to a lower threshold temperature level (e.g., lower than a set target temperature level of the radiofrequency tissue ablation procedure) and then ramps power output back up after temperature reduction and maintenance at the lower threshold temperature level for a certain duration of time, which may allow the tissue to at least partially revascularize and/or rehydrate.

The radiofrequency generator may be configured to transition back to the steady-state phase of operation once a set target temperature has been reached following ramping of the power output back up after temperature reduction to the lower threshold temperature level. In some embodiments, the reduced power output is a power output level at which impedance may still be monitored.

The method may further include accessing the basivertebral nerve within the vertebral body by inserting one or more access instruments within the vertebral body. Inserting the distal end portion of the radiofrequency probe within the vertebral body may include inserting the distal end portion of the radiofrequency probe through at least one of the one or more access instruments.

In accordance with several embodiments, a method of treating back pain (e.g., chronic low back pain) by ablating a basivertebral nerve and/or other intraosseous nerves within one or more vertebral bodies includes inserting at least a distal end portion of a radiofrequency probe within a vertebral body of a subject. The distal end portion of the radiofrequency probe comprises one or more electrodes electrically coupled to a radiofrequency generator. The method also includes applying a thermally conductive gel or a high-viscosity material to at least partially surround the one or more electrodes. The method further includes applying power from the radiofrequency generator to the one or more electrodes to deliver radiofrequency energy sufficient to ablate a basivertebral nerve or other intraosseous nerves within the vertebral body. The thermally conductive gel or high-viscosity material is configured to facilitate directed and/or improved thermal conduction of heat generated by delivery of the radiofrequency energy by the one or more electrodes.

Applying the thermally conductive gel or high-viscosity material may be performed percutaneously after insertion of the distal end of the radiofrequency probe within the vertebral body. Applying the thermally conductive gel or high-viscosity material may alternatively be performed by applying the thermally conductive gel or high-viscosity material prior to insertion of the distal end of the radiofrequency probe within the vertebral body. Applying the thermally conductive gel or high-viscosity material may include coating the one or more electrodes with the thermally conductive gel or high-viscosity material.

The thermally conductive gel or high-viscosity material may completely surround the one or more electrodes (e.g., at least an active electrode of the one or more electrodes if multiple electrodes). Alternatively, the thermally conductive gel or high-viscosity material is positioned only on one side of the one or more electrodes so as to facilitate directed propagation of heat, and therefore, ablation in a particular direction as opposed to omnidirectional propagation of heat.

In accordance with several embodiments, a method of treating chronic low back pain by ablating a basivertebral nerve within one or more vertebral bodies includes inserting at least a distal end portion of a radiofrequency probe within a vertebral body of a subject. The distal end portion of the radiofrequency probe includes one or more electrodes electrically coupled to a radiofrequency generator. The method also includes applying a high-viscosity material to at least partially surround the one or more electrodes and applying power from the radiofrequency generator to the one or more electrodes to deliver radiofrequency energy sufficient to ablate a basivertebral nerve within the vertebral body. The high-viscosity material is configured to facilitate reduction of concentrated heat immediately surrounding the one or more electrodes during delivery of the radiofrequency energy by the one or more electrodes.

Applying the high-viscosity material may be performed percutaneously after insertion of the distal end of the radiofrequency probe within the vertebral body. Alternatively, applying the high-viscosity material may be performed by applying the high-viscosity material prior to insertion of the distal end portion of the radiofrequency probe within the vertebral body. Applying the high-viscosity material may comprise coating the one or more electrodes with the high-viscosity material. In some embodiments, the high-viscosity material completely surrounds the one or more electrodes.

Several embodiments of the invention have one or more of the following advantages: (i) increased treatment accuracy; (ii) increased efficacy and enhanced safety; (iii) increased efficiency as a result of fewer impedance-related stoppages during radiofrequency ablation procedures; (iv) shorter overall duration of radiofrequency ablation procedures; and/or (v) improved user experience and satisfaction.

For purposes of summarizing the disclosure, certain aspects, advantages, and novel features of embodiments of the disclosure have been described herein. It is to be understood that not necessarily all such advantages may be achieved in accordance with any particular embodiment of the disclosure provided herein. Thus, the embodiments disclosed herein may be embodied or carried out in a manner that achieves or optimizes one advantage or group of advantages as taught or suggested herein without necessarily achieving other advantages as may be taught or suggested herein.

The methods summarized above and set forth in further detail below describe certain actions taken by a practitioner; however, it should be understood that they can also include the instruction of those actions by another party. For example, actions such as “applying power” include “instructing the applying of power.” Further aspects of embodiments of the disclosure will be discussed in the following portions of the specification. With respect to the drawings, elements from one figure may be combined with elements from the other figures.

### BRIEF DESCRIPTION OF THE DRAWINGS

Several embodiments of the disclosure will be more fully understood by reference to the following drawings which are for illustrative purposes only:

FIG. 1 is a block diagram illustrating an embodiment of a tissue ablation system.

FIG. 2 is a flow diagram illustrating an embodiment of a method of preventing or mitigating impedance-related stoppages during an ablation procedure.

FIG. 3 is a flow diagram illustrating an embodiment of a method of preventing or mitigating impedance-related stoppages during an ablation procedure.

FIG. 4 is a flow diagram illustrating an embodiment of the impedance limiting phase of the method of FIG. 3.

FIG. 5 is a flow diagram illustrating an embodiment of the secondary impedance limiting phase shown in FIG. 4.

FIG. 6 is a flow diagram illustrating an embodiment of a method of preventing or mitigating impedance-related stoppages during an ablation procedure.

### DETAILED DESCRIPTION

Several embodiments described herein are directed to systems and methods for preventing, mitigating, or reducing the likelihood of occurrence of, impedance-related stoppages during a radiofrequency tissue ablation procedure or other tissue modification procedure or tissue modulation procedure. In some embodiments, an intraosseous nerve (e.g., basivertebral nerve) within a bone (e.g., vertebral body) of the spine is accessed so as to facilitate treatment, or prevention of, back pain (e.g., chronic low back pain originating from one or more vertebral bodies (e.g., nerves innervating one or more endplates and/or discs of one or more vertebral bodies) or other ailments or conditions associated with the spine (e.g., vertebral fractures, spinal tumors, scoliosis, spondylosis). The vertebral body may be located in any level of the vertebral column (e.g., cervical, thoracic, lumbar and/or sacral). Multiple vertebral bodies may be accessed in a single visit or procedure (simultaneously or sequentially). The multiple vertebral bodies may be located in a single spine segment (e.g., two adjacent vertebral bodies in the sacral spine segment (e.g., S1 and S2) or lumbar spine segment (e.g., L3, L4 and/or L5) or thoracic spine segment or cervical spine segment) or in different spine segments (e.g., an L5 vertebra in the lumbar spine segment and an S1 vertebra in the sacral spine segment).

Although described primarily in connection with procedures for the treatment within the spine, the systems and methods may also be used for radiofrequency tissue ablation procedures intended to treat ailments or conditions other than those associated with the spine or back pain. The procedures may involve ablation of nerves outside of bones but related to the spine (e.g., sacroiliac joints, facet joints, etc.). Target treatment locations within bones other than vertebral bodies may also be accessed. For example, target treatment locations within a humerus, radius, femur, tibia, calcaneus, tarsal bones, hips, knees, phalanges, and/or other orthopedic targets may be accessed. The ablation procedures may include, for example, ablation of nerves within or surrounding other bones other than the vertebral column, cardiac tissue ablation for treatment of atrial fibrillation or other abnormal heart rhythms irregularities, tumor ablation at any location within the body (e.g., within bones, lungs, breasts, thyroids, livers, or other organs or tissues), peripheral nerve ablation, pulmonary artery ablation, renal denervation procedures, uterine fibroid ablation, endometrial ablation, and/or the like. The systems and methods described herein may be used in connection with any radiofrequency procedure during which impedance (e.g., tissue impedance or impedance between two electrodes) is monitored.

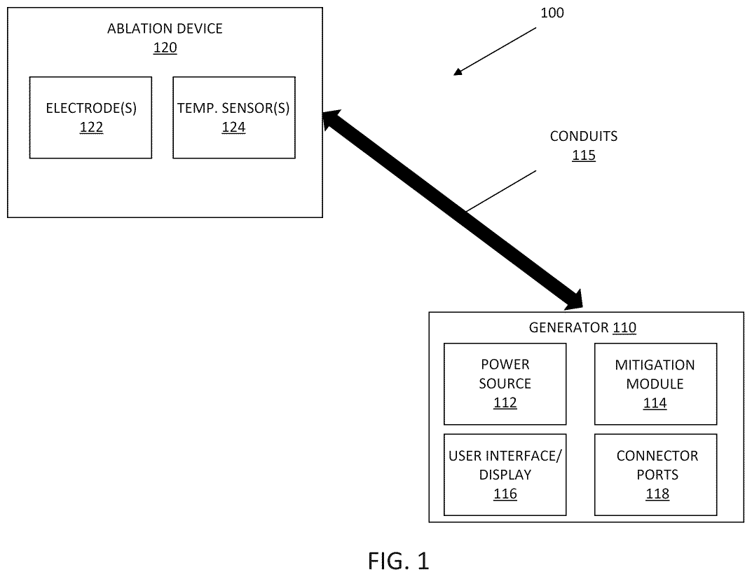

FIG. 1 is a block diagram illustrating an embodiment of a tissue ablation system 100. The tissue ablation system 100 includes a generator 110. The generator 110 may be a radiofrequency generator or other source of ablative radiofrequency energy. The generator 110 is communicatively and/or electrically coupled to an ablation device 120 via one or more conduits 115. The conduits 115 may facilitate bidirectional communication of electrical signals and/or data between the generator 110 and the ablation device 120. For example, the conduits 115 may include one or more electrical wires or lines. The tissue ablation system 100 could alternatively or additionally comprise a tissue modification system adapted to modify tissue in a manner other than ablation (e.g., stimulation, electroporation).

The generator 110 includes a power source 112 adapted to apply radiofrequency energy or power to the ablation device 120 and a mitigation module 114 comprising one or more processors (e.g., implemented in hardware, software, and/or firmware) configured to execute program instructions stored on a non-transitory computer-readable medium (e.g., memory) to monitor temperature, power, and/or impedance in connection with a tissue ablation procedure performed by the tissue ablation system 100. The mitigation module 114 may include hardware circuitry and/or executable software algorithms stored in memory. The one or more hardware processors may be processors of the generator 110 or one or more specific-purpose processors (e.g., microprocessors) dedicated to the mitigation module 114. The generator 110 may also include a user interface and display 116 to facilitate receipt of user input and display of output generated by the mitigation module 114. The generator 110 also includes one, two, three, four or more connector ports 118 to which one or more ablation devices 120 may be connected. In some embodiments, the user interface and display 116 is integrated with the generator 110 (e.g., a touch-screen display and/or integrated user input devices, such as buttons, dials, switches, or knobs). In other embodiments, the user interface and display 116 may be separate from the generator 110 and communicatively and electrically coupled via a wired or wireless connection. User input devices may also include a keyboard, mouse, trackpad, voice-activated input device, or other user input device.

The ablation device 120 may comprise a radiofrequency ablation catheter or probe. The ablation device 120 includes one or more electrodes 122 and one or more temperature sensors 124. The ablation device 120 may include a single monopolar electrode or two electrodes configured to act as a bipolar pair of electrodes. The one or more electrodes 122 may be used to apply or deliver the radiofrequency power or energy generated by the generator 110. The one or more electrodes 122 may additionally be used to monitor tissue impedance (e.g., for safety reasons and/or to reduce the likelihood of tissue desiccation (e.g., charring)). In some embodiments, the same electrode(s) is (are) used to both apply radiofrequency energy and monitor tissue impedance. In other embodiments, the electrode(s) used to apply radiofrequency energy is (are) different than the electrode(s) used to monitor tissue impedance. The one or more temperature sensors 124 may be positioned at one or more locations along the ablation device 120. For example, a temperature sensor may be positioned between two electrodes 122, may be positioned within the one or more electrodes 122, or may be positioned proximal to the one electrode if there is only one electrode or proximal to the proximal-most electrode if multiple electrodes. The ablation device 120 may alternatively or additionally comprise a radiofrequency device adapted to deliver non-ablative radiofrequency energy.

The mitigation module 114 may be configured to receive impedance measurements from the electrodes 122 and temperature measurements from the one or more temperature sensors 124 and may comprise one or more hardware processors configured or programmed to execute one or more algorithms (e.g., stored program instructions) to prevent, mitigate, or reduce the likelihood of, impedance-related stoppages during the radiofrequency ablation procedure, which may advantageously reduce the overall time duration of the radiofrequency ablation procedure. The algorithms may also prevent, mitigate, or reduce the likelihood, of char formation or tissue desiccation, which may be a main cause of treatment-related stoppages. In some embodiments, the mitigation module 114 may be configured to continuously monitor temperature and impedance throughout the radiofrequency ablation procedure, or at least while radiofrequency energy is being applied or delivered.

In some embodiments, during a radiofrequency ablation treatment, the mitigation module 114 of the generator 110 continuously monitors temperature and adjusts power applied by the power source 112 of the generator 110 until the temperature sensor(s) 124 provide signals to the mitigation module 114 indicative of the tissue temperature reaching a preset target temperature (e.g., a temperature between 43 degrees Celsius and 100 degrees Celsius, between 43 degrees Celsius and 70 degrees Celsius, between 60 degrees Celsius and 75 degrees Celsius, between 70 degrees Celsius and 100 degrees Celsius, between 70 degrees Celsius and 85 degrees Celsius, between 65 degrees Celsius and 80 degrees Celsius, between 70 degrees Celsius and 80 degrees Celsius, overlapping ranges thereof, or any value within the recited ranges). If the current temperature is below the target temperature, power is increased at a certain rate to not violate any limits defined in the software specification. If the temperature is above the target temperature, the mitigation module 114 may reduce power, allowing the ablation catheter 120 to cool until the target temperature is met. This temperature-controlled feedback loop is continuously active during a treatment maintaining the target temperature. In some embodiments, continuous comprises real-time or substantially in real-time without pausing. In some embodiments, the feedback may be conducted at periodic intervals.

Applying ablative radiofrequency energy to the target treatment location within the bone may be performed according to the following example treatment parameters: a frequency between 400 kHz and 600 kHz (e.g., between 400 kHz and 500 kHz, between 450 kHz and 500 kHz, between 470 kHz and 490 kHz, between 500 kHz and 600 kHz, overlapping ranges thereof, or any value within the recited ranges); a target temperature of between 43 degrees Celsius and 90 degrees Celsius (e.g., between 43 degrees Celsius and 70 degrees Celsius, between 60 degrees Celsius and 90 degrees Celsius, between 60 degrees Celsius and 80 degrees Celsius, between 65 degrees Celsius and 75 degrees Celsius, between 70 degrees Celsius and 85 degrees Celsius, between 65 degrees Celsius and 80 degrees Celsius, between 70 degrees Celsius and 90 degrees Celsius, overlapping ranges thereof, or any value within the recited ranges); a temperature ramp of between 0.01 and 5 degrees Celsius per second (e.g., between 0.1 and 5 degrees Celsius per second, between 0.1 and 3 degrees Celsius per second, between 0.5 and 3 degrees Celsius per second, between 1 degree and 2.5 degrees Celsius per second, between 1.5 and 4 degrees Celsius per second, between 2 and 5 degrees Celsius per second, between 0.01 and 1 degrees Celsius per second, overlapping ranges thereof, or any value within the recited ranges); and an active energy delivery time of between 1 minute and 20 minutes (e.g., between 1 minute and 5 minutes, between 2 minutes and 8 minutes, between 5 minutes and 15 minutes, between 8 minutes and 12 minutes, between 10 minutes and 20 minutes, overlapping ranges thereof, or any value within the recited ranges). In some embodiments, a target ablation zone is controlled and focused and has a maximum cross-sectional dimension (e.g., major diameter along a long axis) of between 2 mm and 35 mm (e.g., between 2 mm and 30 mm, between 2 mm and 15 mm, between 2 mm and 25 mm, between 10 mm and 30 mm, between 15 mm and 30 mm, between 15 mm and 35 mm, between 5 mm and 20 mm, between 5 mm and 25 mm, overlapping ranges thereof, or any value within the recited ranges) and, if ellipsoidal, a minor diameter along a short axis of between 1 mm and 25 mm (e.g., between 1 mm and 10 mm, between 1 mm and 15 mm, between 5 mm and 15 mm, between 10 mm and 15 mm, between 1 mm and 5 mm, between 10 mm and 25 mm, overlapping ranges thereof, or any value within the recited ranges). In some embodiments, the target ablation zone generally comprises a sphere or approximately a sphere (e.g., a 1 cm sphere) such that the major diameter and minor diameter are equal or substantially equal.

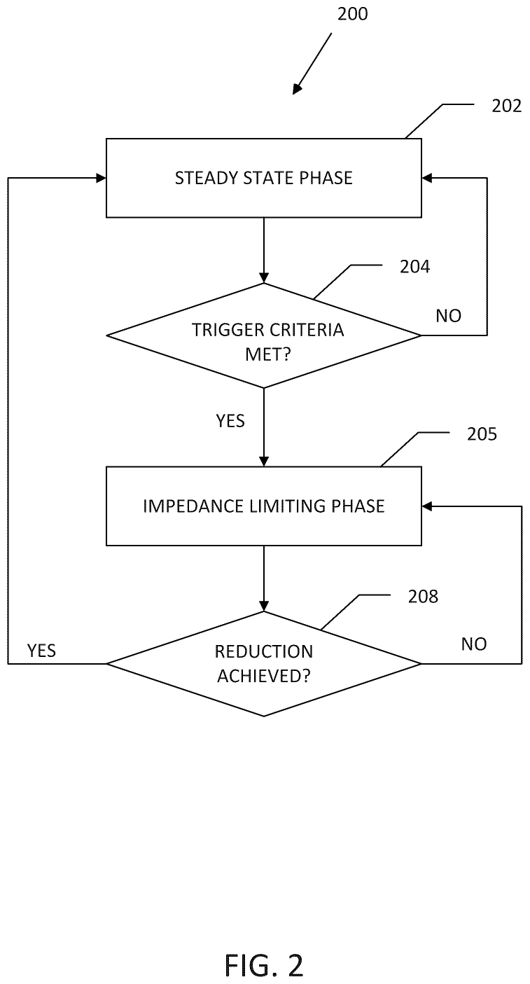

FIG. 2 is a flow diagram illustrating an embodiment of a method 200 of preventing or mitigating impedance-related stoppages during an ablation procedure. In accordance with several embodiments, the impedance-related stoppage mitigation method 200 is configured to detect the start of an impedance event and implement software-based mitigation steps to prevent impedance-based treatment stoppages from occurring. In accordance with several embodiments, the impedance-related stoppage mitigation method 200 advantageously does not require user intervention to operate; however, in some embodiments, an output may be generated for display (e.g., via the user interface and display 116 of the generator 110) to notify a user or operator of its activation.

With reference to Block 202, the method 200 starts in a steady state phase in which the mitigation module 114 executes stored program instructions that involve continuously monitoring impedance magnitude changes and a rate of change of the impedance, based on impedance measurements received from the ablation device 120 (e.g., from the one or more electrodes 122 of a radiofrequency probe). If certain predetermined or preset trigger criteria are met (at Decision Block 204), the method 200 enters an impedance limiting phase at Block 205. The trigger criteria may include trigger levels based on temperature and/or trigger limits based on impedance changes (e.g., magnitude and/or rate of change). In some embodiments, only rate of change is monitored and magnitude is not monitored. In some embodiments, quality of contact between the electrodes and tissue is not assessed.

If one or more trigger criteria are met (e.g., a potential impedance violation is detected), the mitigation module 114 in the impedance limiting phase at Block 205 momentarily reduces the target temperature, which in turn causes the mitigation module 114 to reduce power output provided by the power source 112 of the generator 110. The power reduction allows the probe temperature to decrease to a specific lower threshold value and then dwell at the lower threshold value for a duration of time. In accordance with several embodiments, temperature reduction and dwell duration advantageously allows the tissue to “revascularize” (e.g., be refreshed with blood flow) and potentially rehydrate desiccated tissue closest to the electrode(s) 122 of the ablation device 120. Return of blood flow may also remove, to a certain extent, coagulum and char that may have formed.

With reference to decision block 208, once reduction (e.g., maintenance) of temperature and impedance have been achieved at a lower threshold level (e.g., lower temperature than a set target temperature level of the radiofrequency tissue ablation procedure), the method 200 exits the impedance limiting phase and returns to the steady state phase. The mitigation module 114 (e.g., software executed by one or more hardware processors) continues monitoring impedance and, if another impending impedance event is detected, the mitigation module 114 will transition to the impedance limiting phase to mitigate and/or prevent any impedance-based treatment stoppages.

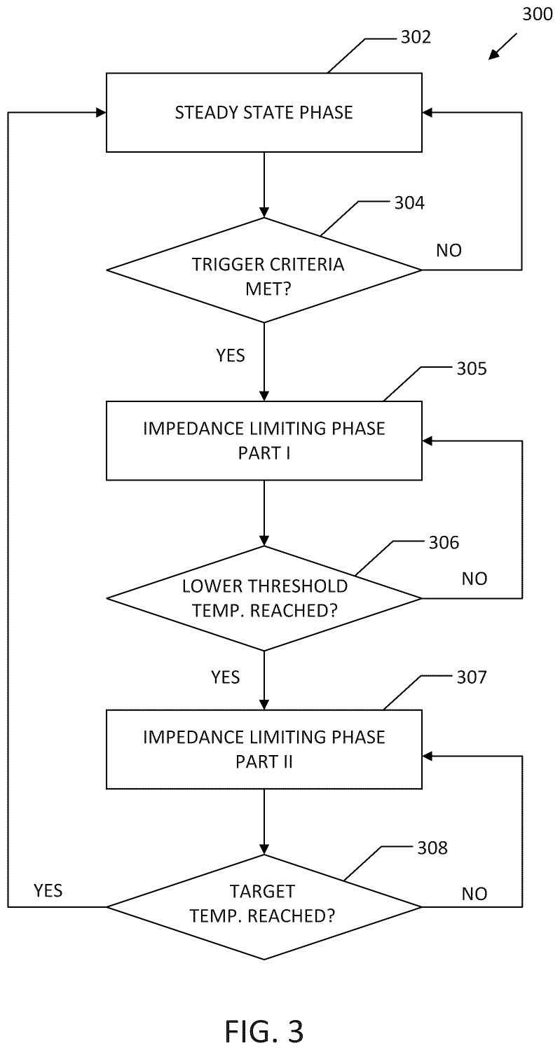

FIG. 3 is a flow diagram illustrating an embodiment of a method 300 of preventing or mitigating impedance-related stoppages during an ablation procedure. The method 300 of FIG. 3 may be similar to method 200 but may include additional details or steps. Similar to method 200, the method 300 begins in the steady state phase at Block 302 and remains in the steady state phase until certain trigger criteria are met (Decision Block 304). In the steady state phase, the mitigation module 114 monitors rate of change of impedance by taking a periodic impedance reading at a certain interval (e.g., every 5 ms-100 ms, 5 ms-50 ms, 5 ms-15 ms, 10 ms-30 ms, 15 ms-40 ms, 20 ms-50 ms, 15 ms-30 ms, 30 ms-50 ms, 50 ms-100 ms, overlapping ranges thereof, or any value within the recited ranges). The mitigation module 114 calculates the rate of change of impedance by taking a current impedance value and comparing with (e.g., subtracting from) previous impedance reading values. In some embodiments, the monitoring of rate of change is continuously performed substantially in real time.

In some embodiments, the trigger criteria (e.g., impedance rate of change monitoring) are not evaluated or monitored during an initial ramp phase of the generator 110 (e.g., initial increase of temperature to a target temperature). In other embodiments, the trigger criteria are evaluated during the initial ramp phase. The mitigation module 114 calculates a rolling average of a certain number of data points of impedance rate of change values. For example, an oldest data point is replaced with a new data point at a time each new data point is received). The certain number of data points for which a rolling average is calculated may be between 5 and 200 (e.g., between 5 and 100, between 5 and 20, between 5 and 10, between 10 and 40, between 15 and 45, between 20 and 50, between 25 and 75, between 40 and 80, between 50 and 100, between 20 and 80, between 25 and 75, between 5 and 50, between 50 and 150, between 100 and 200, overlapping ranges thereof, or any value within the recited ranges).

The method 300 may include an anti-bump (or anti-spike) prevention condition that requires that the trigger criteria (e.g., impedance rate of change threshold level) is/are violated or triggered for a certain number of data points (e.g., more than 1-50 data points, 5-50 data points, 5-20 data points, 10-30 data points, 20-40 data points, 30-50 data points, 25-35 data points, overlapping ranges thereof, or any value within the recited ranges) and/or a certain duration of time (e.g., 5 ms-5000 ms, 5 ms-100 ms, 5 ms-50 ms, 10 ms-40 ms, 20 ms-60 ms, 30 ms-80 ms, 50 ms-100 ms, 60 ms-90 ms, 100 ms-500 ms, 250 ms-750 ms, 500 ms-1000 ms, 1000 ms-2500 ms, 1500 ms-3000 ms, 2000 ms-4000 ms, 2500 ms-5000 ms, 3000 ms-5000 ms, 4000 ms-5000 ms, overlapping ranges thereof, or any value within the recited ranges) consecutively before the mitigation module 114 will allow the trigger criteria to be met and enable the transition to a first part of an impedance limiting phase at Block 305. In some embodiments, if the rolling average is above 0-100 (e.g., 0-5, 1-8, 2-6, 3-9, 0-50, 10-40, 20-60, 30-90, 40-80, 50-100, overlapping ranges thereof, or any value within the recited ranges), and passes the anti-bump, or anti-spike, test, the method 300 is allowed to enter the first part of the impedance limiting phase. The units of the rolling average of rate of impedance change over time may be ohms per seconds squared.

At Block 305 in the first part of the impedance limiting phase, the mitigation module 114 reduces power to the one or more electrodes 122 of the ablation device 120 to a reduced level (e.g., a minimum level that allows impedance monitoring to continue) at which no significant energy (e.g., energy insufficient to increase thermal heating) is delivered to the patient. During this temperature “freefall”, the temperature naturally falls and is monitored by the mitigation module 114 while an overall radiofrequency ablation procedure timer displayed on the user interface/display 116 is paused. In some embodiments, the timer blinks or otherwise indicates that the impedance mitigation method is active.

With reference to decision Block 306, the mitigation module 114 determines whether the current monitored temperature (as indicated by signals received from the temperature sensor(s) 124) reaches a lower threshold temperature (e.g., temperature between 43 degrees Celsius and 100 degrees Celsius, between 43 degrees and 70 degrees Celsius, between 43 degrees Celsius and 67 degrees Celsius, between 50 degrees Celsius and 60 degrees Celsius, between 60 degrees Celsius and 65 degrees Celsius, between 60 degrees Celsius and 90 degrees Celsius, between 70 degrees Celsius and 100 degrees Celsius, overlapping ranges thereof, or any value within the recited ranges). If the current temperature does not reach the lower threshold temperature, the mitigation module 114 continues to operate the ablation device 120 in the first part of the impedance limiting phase at the reduced power level and continues monitoring the current temperature. Once the current temperature reaches the lower threshold temperature, the mitigation module 114 proceeds to a second part of the impedance limiting phase (Block 307) and causes the power source 112 to apply power from the generator 110 to the ablation device 120 to maintain the temperature at or near the lower threshold temperature.

At Block 307, the mitigation module 114 may hold at the lower threshold temperature for a dwell period (e.g., between greater than 0 and less than or equal to 200 seconds, between 1 and 20 seconds, between 5 and 30 seconds, between 10 and 45 seconds, between 15 and 60 seconds, between 45 and 90 seconds, between 60 and 120 seconds, between 100 and 200 seconds, overlapping ranges thereof, or any value within the recited ranges). Once the dwell period has expired, the mitigation module 114 may ramp back to the set target temperature using a ramp rate of 0.01-5 degrees Celsius per second (e.g., 0.01-0.10 degrees Celsius per second, 0.05-1.5 degrees Celsius per second, 0.10 to 2.0 degrees Celsius per second, 0.1 to 1. 0 degrees Celsius per second, 1.0 to 5.0 degrees Celsius per second, overlapping ranges thereof, or any value within the recited ranges). Although both the hold and ramp up steps are included in the second part of the impedance limiting phase, they may be considered two separate subparts (e.g., second and third parts, respectively).

Once the measured temperature reaches the set target temperature (decision Block 308), the mitigation module 114 exits the impedance phase and proceeds back to the steady state phase (Block 302) to complete the treatment. In some embodiments, the method 300 is not allowed to re-enter the first part of the impedance limiting phase at Block 305 until after a particular duration of time has elapsed after returning to the steady state phase (e.g., between greater than 0 and less than or equal to 200 seconds, between 1 and 20 seconds, between 5 and 30 seconds, between 10 and 45 seconds, between 15 and 60 seconds, between 45 and 90 seconds, between 60 and 120 seconds, between 100 and 200 seconds, overlapping ranges thereof, or any value within the recited ranges).

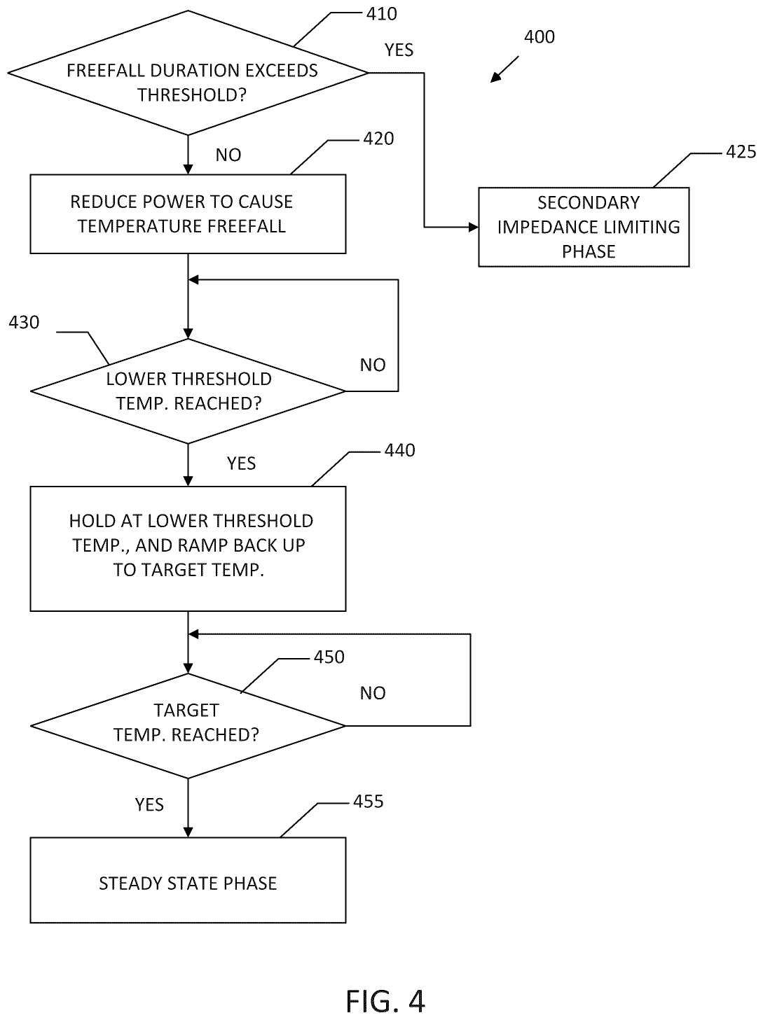

FIG. 4 is a flow diagram illustrating an embodiment of the impedance limiting phase of the method 300 of FIG. 3 (e.g., steps involved at Blocks 305, 306, 307 and 308). With reference to decision Block 410, the mitigation module 114 may keep track of the overall time spent in a temperature freefall portion of the impedance limiting phase (e.g., Part 1 of the impedance limiting phase described in connection with FIG. 3) during a particular radiofrequency tissue ablation procedure. If the total freefall duration for the procedure exceeds a threshold duration (e.g., greater than 0 minutes to less than or equal to 15 minutes, 1-15 minutes, 1-5 minutes, 2-8 minutes, 3-10 minutes, 5-15 minutes, overlapping ranges thereof, or any value within the recited ranges), the mitigation module 114 executes a secondary impedance limiting phase (Block 425) for any future impedance events or triggers. If the total overall time spent in a temperature freefall portion does not exceed the threshold duration, then the method 400 proceeds to Block 420. In some embodiments, the method 400 continues with the current impedance limiting phase with no changes if the overall time spent in the temperature freefall portion exceeds the threshold duration while the impedance limiting phase is in process. At Block 420, the mitigation module 114 reduces power (e.g., to a minimum level that allows impedance monitoring to continue, but no significant energy (e.g., energy insufficient to cause thermal heating) to be delivered to the patient), thereby causing a temperature “freefall.” During this temperature “freefall”, the temperature naturally falls and is monitored by the mitigation module 114 (e.g., via the temperature sensor(s) 124 of the ablation device 120).

With reference to decision Block 430, the mitigation module 114 determines whether the temperature reaches the lower threshold temperature, as discussed previously in connection with method 300 of FIG. 3. If the temperature does not reach the lower threshold temperature, the mitigation module continues operating the ablation device 120 at the reduced power level and continues monitoring the temperature. Once the current temperature reaches the lower threshold temperature, the mitigation module 114 (at Block 440) instructs the power source 112 to provide sufficient power to the ablation device 120 to maintain the temperature at the lower threshold temperature. The stored program instructions executed by the mitigation module 114 cause the ablation system 100 to hold at the lower threshold temperature for the dwell period (as described previously). Once the dwell period has expired, the mitigation module 114 increases power to the one or more electrodes, using the ramp rate (as also described previously), to increase the temperature to the set target temperature. With reference to decision Block 450, the mitigation module 114 monitors the temperature to determine whether the temperature reaches the target temperature. Once the target temperature is reached again, the mitigation module 114 exits the impedance limiting phase and proceeds back to the steady state phase at Block 455 to complete the treatment. As discussed previously, the impedance limiting phase may not be allowed to be re-entered for a predetermined duration after being exited.

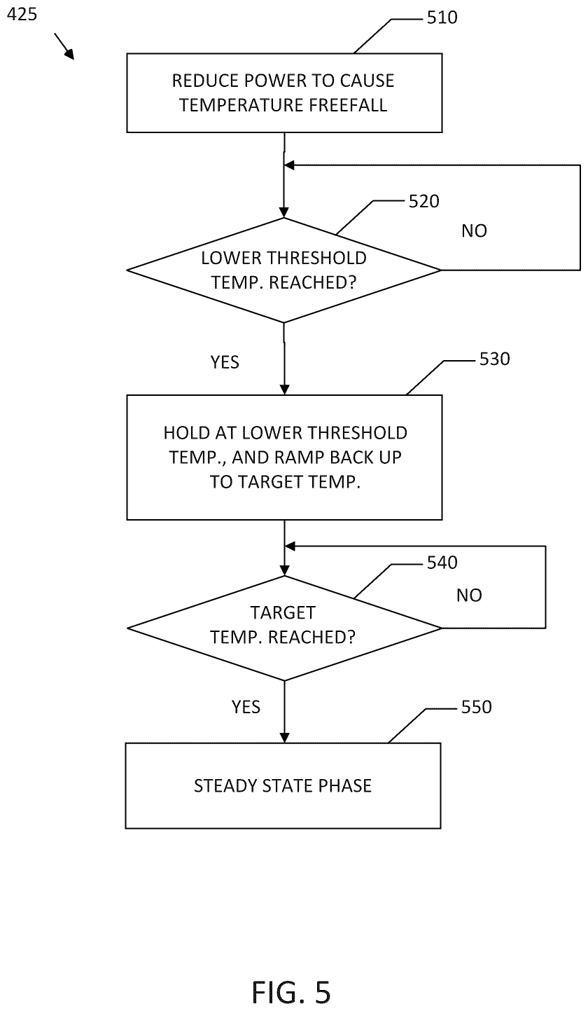

FIG. 5 is a flow diagram illustrating an embodiment of the secondary impedance limiting phase 425 shown in FIG. 4. For example, if the freefall duration time expires, the mitigation module 114 may execute a secondary set of program instructions (e.g., involving a second, different set of parameters) for the secondary impedance limiting phase 425. In some embodiments, the secondary impedance limiting phase 425 includes the same procedural steps as the primary impedance limiting phase but using different parameter values for at least some of the parameters. At Block 510, the mitigation module 114 may cause a reduction of power output (e.g., to a minimum level allowing impedance readings, but no significant energy to be delivered to the patient sufficient to cause thermal heating), thereby causing the temperature freefall as described previously. During this temperature freefall, the temperature naturally falls and is monitored by the mitigation module 114 (e.g., via the temperature sensor(s) 124 of the ablation device 120). In some embodiments, the timer displayed on the user interface/display 116 does not stop or blink during the secondary impedance limiting phase 425.

With reference to decision Block 520, the mitigation module 114 determines whether the temperature reaches a lower threshold temperature (e.g., temperature between 43 degrees Celsius and 70 degrees Celsius, between 43 degrees Celsius and 67 degrees Celsius, between 50 degrees Celsius and 60 degrees Celsius, between 60 degrees Celsius and 65 degrees Celsius, between 43 degrees Celsius and 100 degrees Celsius, overlapping ranges thereof, or any value within the recited ranges). If the temperature does not reach the lower threshold temperature, the mitigation module 114 continues operating the ablation device at the reduced power level and continues monitoring the temperature. Once the temperature reaches the lower threshold temperature, the mitigation module 114 (at block 530) causes the power source 112 to provide sufficient power to the ablation device 120 to maintain the temperature at or near the lower threshold temperature. The temperature is caused to be held at the lower threshold temperature for a dwell period (e.g., between greater than 0 and less than or equal to 200 seconds, between 1 and 20 seconds, between 5 and 30 seconds, between 10 and 45 seconds, between 15 and 60 seconds, between 45 and 90 seconds, between 60 and 120 seconds, between 100 and 200 seconds, overlapping ranges thereof, or any value within the recited ranges). Once the dwell period has expired, the mitigation module 114 may ramp temperature back to the normal target temperature using a predetermined ramp rate, as described previously.

With reference to decision Block 540, the mitigation module 114 monitors the temperature to determine whether the temperature reaches the target temperature. Once the target temperature is reached, the mitigation module 114 exits the secondary impedance limiting phase and proceeds back to the steady state phase at Block 550 to complete the treatment.

### Thermally Conductive Gels or Non-Software-Based Approaches

The prevention, mitigation, or reduction of likelihood of impedance-related stoppages during a radiofrequency ablation procedure may also be supplemented or replaced by non-software-based approaches. For example, thermally conductive gels or high-viscosity materials may be used to facilitate thermal conduction, and thus reduce impedance spikes or bumps that may cause impedance-based stoppages.

During a thermal ablation procedure, heat generated by application of radiofrequency energy to tissue surrounds the electrode(s) 122 of the ablation device 120 because tissue thermal conductivity may generally be poor in certain target locations. Poor tissue thermal conductivity can lead to very steep temperature gradients radiating from the electrode(s) 122 and can quickly desiccate the tissue closest to the electrode(s) 122, thereby causing high impedance and subsequent impedance-based treatment stoppages. Furthermore, the thermal dose necessary to achieve optimal tissue ablation typically does not radiate more than a certain depth (e.g., about two centimeters or other depths depending on the tissue) into the tissue.

One method to overcome poor tissue conductivity is to modulate the tissue characteristics by modifying the tissue's thermal conductivity. By administering (e.g., percutaneously administering) a thermally conductive gel on or near the electrode(s) 122 of the ablation device (e.g., radiofrequency probe) 120, heat can more easily pass through the conductive gel, thereby heating tissue further away from the electrode(s) 122 and increasing depth of penetration. Furthermore, the gel may reduce the buildup of heat and high temperatures on the electrode surfaces and tissue closest to the electrode(s) 122, thereby reducing tissue desiccation and potentially preventing impedance-based treatment stoppage.

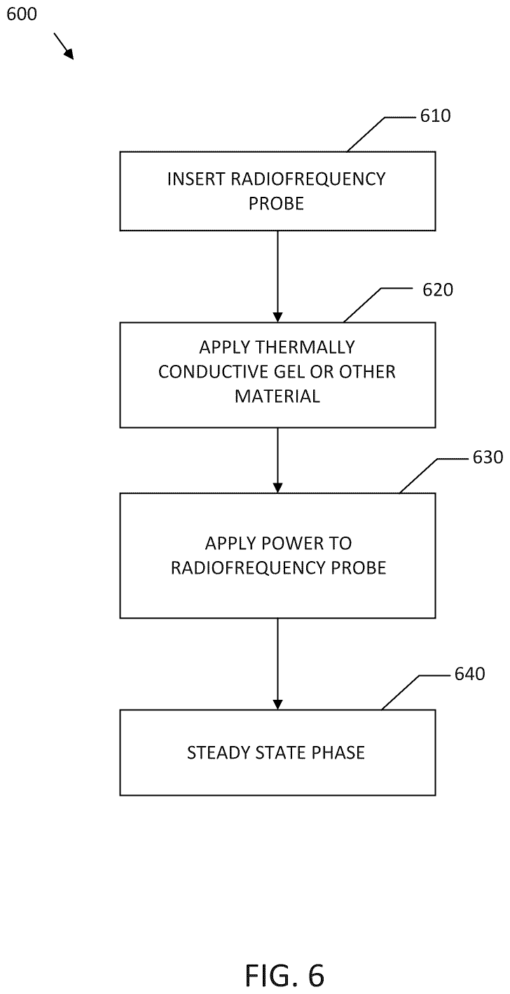

FIG. 6 is a flow diagram illustrating an embodiment of a method 600 of preventing or mitigating impedance-related stoppages during an ablation procedure. At Block 610, an ablation device comprising a radiofrequency probe is inserted into a vertebral body. In some embodiments, the radiofrequency probe is generally similar to the radiofrequency ablation catheter or probe 120 described above in connection with FIG. 1. For example, the radiofrequency probe includes one or more electrodes that are electrically connected to a generator and are configured to receive radiofrequency energy from a power source within the generator. The electrodes are configured to deliver the radiofrequency energy from the power source to a target ablation location within the vertebral body. In some embodiments, the radiofrequency probe is inserted into the vertebral body through an access instrument that is inserted into the vertebral body. In some embodiments, the radiofrequency probe is configured to also function as an access instrument.

At Block 620, a thermally conductive gel or other material (e.g., high-viscosity material) is administered to the one or more electrodes on the radiofrequency probe or to the target ablation location within the vertebral body. In some embodiments, the gel or other material is administered through a lumen of the same access instrument through which the ablation device 120 is inserted. In some embodiments, a separate access instrument is used to administer the thermally conductive gel or high-viscosity material. In these embodiments, the separate access instrument may be inserted through the same incision as the incision in which the radiofrequency probe was inserted or may be inserted into a different incision.

In some embodiments, the thermally conductive gel or high-viscosity material can be administered such that the gel or other material at least partially surrounds one or more of the electrodes on the radiofrequency probe. More specifically, the thermally conductive gel or other material can be administered on either side of the electrode(s) on the radiofrequency probe such that the gel or other material is within a sufficiently close vicinity to the electrodes to transfer heat to the target ablation location. Administering the thermally conductive gel or other material in this way allows for the thermally conductive gel to direct or focus heat generated by the electrodes in a particular direction so as to preferentially heat an area of interest within the tissue. In some embodiments, the thermally conductive gel or other material can be administered such that it completely surrounds the one or more electrodes on the radiofrequency probe. In embodiments where multiple vertebral bodies are accessed simultaneously using multiple radiofrequency probes, the thermally conductive gel or other material may be administered such electrodes on each of the multiple radiofrequency probes are at least partially surrounded by the thermally conductive gel or other material.

In the illustrated embodiment, the thermally conductive gel or other material is administered after the radiofrequency probe is inserted into the vertebral body. In some embodiments, however, the thermally conductive gel or other material is administered before the probe is inserted. For example, in some embodiments, the electrodes on the radiofrequency probe are coated with the thermally conductive gel prior to radiofrequency probe being inserted into the body of a subject. In some embodiments, the thermally conductive gel or other material is administered adjacent to the one or more electrode(s) along a distal end portion of the radiofrequency probe prior to insertion of the probe within a body of the subject. In some embodiments, the thermally conductive gel or other material may be administered simultaneously with the radiofrequency probe being inserted into the body of the subject. In some embodiments, Blocks 610 and 620 can be reversed such that thermally conductive gel or other material is inserted into the vertebral body prior to insertion of the radiofrequency probe.

At optional Block 630, the radiofrequency generator begins to apply power to the one or more electrodes on the radiofrequency probe. As explained above in connection with FIG. 3, the radiofrequency generator can be initially run in an initial ramp phase in which the power output by the radiofrequency generator increases over time until a measured temperature reaches a target temperature. At this point, at Block 640, the system can operate in a steady-state phase. In some embodiments, the steady state phase can be generally similar to the steady state phase described above in connection with FIGS. 2-5. The method 600 may also be performed in a location other than a vertebral body (e.g., another bone or non-bone location).

The method 600 to reduce or prevent impedance-based treatment stoppages may involve use of a high-viscosity material such as a SURGICEL® hemostat or similar absorbable hemostat to stop or slow blood flow around or near the electrode(s) 122 of the ablation device 120 (e.g., radiofrequency probe). The high-viscosity material may be in a powder form or a fluid form. The high-viscosity material can have a viscosity in a range sufficient to reduce blood flow (e.g., between 2500 cP and 7500 cP, between 2500 cP and 10,000 cP, between 5000 cP and 15,000 cP, between 5000 cP and 10,000 cP, overlapping ranges thereof, or any value within the recited ranges).

In accordance with several embodiments, the generator 110 is a temperature-controlled radiofrequency ablation system. The generator 110 controls the temperature at an active tip of the ablation device 120 by constantly modulating power output to maintain the target temperature. Blood flow acts as a heat sink and reduces the volume of tissue heated to the target temperature either through large blood vessels or capillary-mediated perfusion. If the ablation device 120 is placed near or on a blood vessel, the excess blood flow will wick away heat quickly causing the generator 110 to increase power output in order to reach and maintain the target temperature. The increase in power output to maintain target temperature in the presence of blood flow can cause the formation of coagulum or char (desiccated tissue) on the electrode(s) 122 of the ablation device 120, leading to a high impedance-based treatment stoppage. Adding a high viscosity material to the target treatment location can slow or stop blood flow which may prevent, or reduce the likelihood of, a premature treatment stoppage. In some embodiments, the high-viscosity material may be administered percutaneously before the insertion of the ablation device 120. In some embodiments, the high-viscosity material may be administered percutaneously after the insertion of the ablation device. In embodiments where an access instrument is used to insert the ablation device 120, the access instrument may also be used to administer the high-viscosity material. For example, in some embodiments, the high-viscosity material may be administered through a lumen of an access instrument through which the ablation device 120 is inserted or through. In some embodiments, however, a separate access instrument is used to administer the high-viscosity material than is used to insert the ablation device 120. In these embodiments, the separate access instrument may be inserted through either the same incision as the first access instrument or may be inserted a different incision as the first access instrument. In some embodiments, the high-viscosity material may be coated along the one or more electrode(s) 122 and/or adjacent the one or more electrode(s) 122 along the distal end portion of the ablation device 120 prior to insertion of the ablation device 120 within a body of the subject. A portion of the one or more electrodes (122) or one side of the ablation device 120 may be coated or surrounded so as to preferentially direct thermal conduction and heating in a particular direction.

### Additional System Embodiments and Non-Limiting Examples

The tissue ablation system 100 may also include one or more access tools to facilitate access to a treatment location (e.g., an intraosseous location, such as a location within a vertebral body). Access tools may include an introducer assembly including an outer cannula and a sharpened stylet, an inner cannula configured to be introduced through the outer cannula, and/or one or more additional stylets, curettes, or drills to facilitate access to an intraosscous location within a vertebral body or other bone. The access tools (e.g., outer cannula, inner cannula, stylets, curettes, drills) may have pre-curved distal end portions or may be actively steerable or curveable. Any of the access tools may have beveled or otherwise sharp tips or they may have blunt or rounded, atraumatic distal tips. Curved drills may be used to facilitate formation of curved access paths within bone. Any of the access tools may be advanced over a guidewire in some embodiments.

In some embodiments, an outer cannula assembly (e.g., introducer assembly) includes a straight outer cannula and a straight stylet configured to be received within the outer cannula. The outer cannula assembly may be inserted first to penetrate an outer cortical shell of a bone and provide a conduit for further access tools to the inner cancellous bone. An inner cannula assembly may include a cannula having a pre-curved or steerable distal end portion and a stylet having a corresponding pre-curved or steerable distal end portion. Multiple stylets having distal end portions with different curvatures may be provided in a kit and selected from by a clinician. The inner cannula assembly may alternatively be configured to remain straight and non-curved.

In some embodiments, the tissue ablation system 100 comprises various features that are present as single features (as opposed to multiple features). For example, in one embodiment, the ablation device includes a single electrode. A single temperature sensor 124 (thermocouple or other means for measuring temperature) may also be included. Multiple features or components are provided in alternate embodiments. For example, multiple ablation devices 120 may be coupled to the generator 110 and the ablation device(s) 120 may include multiple electrodes 122 and/or multiple temperature sensors 124.

The generator 110 can include multiple engines or modules for performing the processes and functions described herein, such as the mitigation module 114 described above. The engines or modules can include programmed instructions for performing processes as discussed herein. The programming instructions can be stored in a memory. The programming instructions can be implemented in C, C++, JAVA, or any other suitable programming languages. In some embodiments, some or all of the portions of the mitigation module 114 can be implemented in application specific circuitry such as ASICs and FPGAs.

Any methods described herein may be embodied in, and partially or fully automated via, software code modules (e.g., in the form of an algorithm or machine readable instructions) stored in a memory or tangible, non-transitory computer-readable medium executed by one or more processors or other computing devices. The one or more processors or other computing devices may be located within the generator 110 or stored on a remote server and accessed by a communications interface of the generator 110. The software may be downloaded to a processor in electronic form. In embodiments involving multiple processors, the processors may operate in parallel to form a parallel processing system in which a process is split into parts that execute simultaneously on different processors of the ablation system. The methods may be executed on the computing devices (e.g., the generator 110) in response to execution of software instructions or other executable machine-readable code read from a tangible computer readable medium. A tangible computer readable medium is a data storage device that can store data that is readable by a computer system. Examples of computer readable mediums include read-only memory (e.g., ROM or PROM, EEPROM), random-access memory, other volatile or non-volatile memory devices, CD-ROMs, magnetic tape, flash drives, and optical data storage devices. The modules described herein (for example, the mitigation module 114) may comprise structural hardware elements and/or non-structural software elements stored in memory (for example, algorithms or machine-readable instructions executable by processing or computing devices).

In addition, embodiments may be implemented as computer-executable instructions stored in one or more tangible computer storage media. As will be appreciated by a person of ordinary skill in the art, such computer-executable instructions stored in tangible computer storage media define specific functions to be performed by computer hardware such as computer processors. In general, in such an implementation, the computer-executable instructions are loaded into memory accessible by at least one computer processor (for example, a programmable microprocessor or microcontroller or an application specific integrated circuit). The at least one computer processor then executes the instructions, causing computer hardware to perform the specific functions defined by the computer-executable instructions. As will be appreciated by a person of ordinary skill in the art, computer execution of computer-executable instructions is equivalent to the performance of the same functions by electronic hardware that includes hardware circuits that are hardwired to perform the specific functions. As such, while embodiments illustrated herein are typically implemented as some combination of computer hardware and computer-executable instructions, the embodiments illustrated herein could also be implemented as one or more electronic circuits hardwired to perform the specific functions illustrated herein.

Although certain embodiments and examples have been described herein, aspects of the methods and devices shown and described in the present disclosure may be differently combined and/or modified to form still further embodiments. Optional features of various device and system embodiments may be included in some embodiments and not in others. Additionally, the methods described herein may be practiced using any device suitable for performing the recited steps. Further, the disclosure (including the figures) herein of any particular feature, aspect, method, property, characteristic, quality, attribute, element, or the like in connection with various embodiments can be used in all other embodiments set forth herein. Any section headings used herein are merely provided to enhance readability and are not intended to limit the scope of the embodiments disclosed in a particular section to the features or elements disclosed in that section.

While the embodiments are susceptible to various modifications, and alternative forms, specific examples thereof have been shown in the drawings and are herein described in detail. It should be understood, however, that the embodiments are not to be limited to the particular forms or methods disclosed, but to the contrary, the embodiments are to cover all modifications, equivalents, and alternatives falling within the spirit and scope of the various embodiments described and the appended claims. Any methods disclosed herein need not be performed in the order recited and in other alternative embodiments one or more method steps may be skipped altogether. The methods disclosed herein include certain actions taken by a practitioner; however, they can also include any third-party instruction of those actions, either expressly or by implication.

Various embodiments of the disclosure have been presented in a range format. It should be understood that the description in range format is merely for convenience and brevity and should not be construed as an inflexible limitation on the scope of the invention. The ranges disclosed herein encompass any and all overlap, sub-ranges, and combinations thereof, as well as individual numerical values within that range. For example, description of a range such as from 70 to 115 degrees should be considered to have specifically disclosed subranges such as from 70 to 80 degrees, from 70 to 100 degrees, from 70 to 110 degrees, from 80 to 100 degrees etc., as well as individual numbers within that range, for example, 70, 80, 90, 95, 100, 70.5, 90.5 and any whole and partial increments therebetween. Language such as “up to,” “at least,” “greater than,” “less than,” “between,” and the like includes the number recited. Numbers preceded by a term such as “about” or “approximately” include the recited numbers. For example, “approximately 30-50%” includes 30% and 50%. The terms “generally” and “substantially” as used herein represent an amount close to the stated amount that still performs a desired function or achieves a desired result. As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “70” is disclosed, then “about 70” is also disclosed. Any numerical range recited herein is intended to include all sub-ranges subsumed therein.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

What is claimed is:

1. A radiofrequency tissue ablation system, for use with a radiofrequency probe including one or more electrodes and one or more temperature sensors, the system configured to reduce occurrences of impedance-related stoppages of radiofrequency energy delivery during a radiofrequency tissue ablation procedure, the system comprising:
a radiofrequency generator comprising a power source electrically coupled to the one or more electrodes and one or more processors operatively coupled to the power source and to the one or more temperature sensors,
wherein the one or more processors are configured to:
cause the power source to deliver radiofrequency energy to the one or more electrodes, the radiofrequency energy being sufficient to provide controlled heating of tissue surrounding the one or more electrodes; and
receive a plurality of temperature measurements from the one or more temperature sensors, calculate a magnitude and a rate of change of tissue impedance based on signals received from the one or more electrodes and determine whether one or more trigger criteria are met;
if the one or more trigger criteria are met, cause the power source to temporarily reduce power output to reduce temperature to a lower threshold temperature and to ramp power output back up after achieving the lower threshold temperature.

2. The system of claim 1, wherein the radiofrequency probe comprises two electrodes positioned along a distal end portion of the radiofrequency probe configured to operate as a bipolar pair of electrodes.

3. The system of claim 2, wherein the radiofrequency energy has a frequency between 400 kHz and 600 kHz and is applied for a duration sufficient to generate a controlled ablation zone to ablate tissue within the controlled ablation zone.

4. The system of claim 3, wherein the duration is between 5 minutes and 15 minutes.

5. The system of claim 4, wherein the ablated tissue comprises a basivertebral nerve within a vertebral body.

6. The system of claim 1, wherein the signals received from the one or more electrodes are received at a certain interval, and wherein the interval is between 5 ms and 100 ms.

7. The system of claim 1, wherein the calculation of the rate of change of tissue impedance involves calculation of a rolling average of tissue impedance measurements over a particular time duration.

8. The system of claim 7, wherein the particular time duration is between 5 ms and 20,000 ms.

9. The system of claim 1, wherein the one or more trigger criteria include at least one criterion based on temperature and at least one criterion based on the rate of change of tissue impedance.

10. The system of claim 9, wherein the one or more trigger criteria include a predetermined upper threshold rate of change of tissue impedance.

11. The system of claim 10, wherein the one or more trigger criteria have to be met for a certain number of data points or for a certain duration of time before the transition to the impedance-limiting phase of operation.

12. The system of claim 11, wherein the certain number of data points is between 1 and 50.

13. The system of claim 11, wherein the certain duration of time is between 5 ms and 5000 ms.

14. The system of claim 1, wherein the power output is reduced until the lower threshold temperature is reached.

15. The system of claim 14, wherein, when in the impedance-limiting phase of operation, the one or more processors are configured to cause the temperature to remain at the lower threshold temperature for a dwell period and wherein the ramp up in power does not occur until after the dwell period has passed.

16. The system of claim 15, wherein the one or more processors are configured to transition the radiofrequency generator back to the steady-state phase of operation after a pre-set target temperature has been reached following the ramp up in power.

17. A radiofrequency tissue ablation system, for use with a radiofrequency probe including one or more electrodes and one or more temperature sensors, the system configured to reduce occurrences of impedance-related stoppages of radiofrequency energy delivery during a radiofrequency tissue ablation procedure, the system comprising:
a radiofrequency generator comprising a power source electrically coupled to the one or more electrodes and one or more processors operatively coupled to the power source and to the one or more temperature sensors,
wherein the one or more processors are configured to:
cause the power source to deliver radiofrequency energy to the one or more electrodes, the radiofrequency energy being sufficient to provide controlled heating of tissue surrounding the one or more electrodes; and
receive a plurality of temperature measurements from the one or more temperature sensors, monitor tissue impedance based on signals received from the one or more electrodes and determine whether one or more trigger criteria are met;
if the one or more trigger criteria are met, cause the power source to temporarily modify power output to control temperature and subsequently adjust power output.

18. The system of claim 17, wherein monitoring tissue impedance comprises calculating a rate of change of tissue impedance based on a rolling average of impedance measurements, and wherein the one or more trigger criteria include a predetermined threshold rate of change of tissue impedance.

19. The system of claim 17, wherein temporarily modifying power output comprises reducing power output to a minimum level sufficient to maintain impedance monitoring capability while preventing significant energy delivery, and wherein subsequently adjusting power output comprises increasing power output after maintaining a reduced temperature for a predetermined dwell period.

20. The system of claim 17, wherein the system is configured to prevent re-entry into the impedance-limiting phase of operation for a predetermined duration after transitioning back to the steady-state phase of operation, and wherein the predetermined duration is between 5 seconds and 200 seconds.
